SEQUENCE LISTING (7 sequences)

SEQ ID NO: 1 - DNA, 43 bp, Homo sapiens
cgtgctcccctcaggcgtccgcgcaccagggccaccgtgtccc

SEQ ID NO: 2 - DNA, 46 bp, Homo sapiens
tctggccagggaacgtggaaggcgcaccgacagggatccggccagg

SEQ ID NO: 3 - DNA, 53 bp, Homo sapiens
aggctgccagacggactttctgcggcgcgcaagtgatgcccggcgcaggcaga

SEQ ID NO: 4 - DNA, 52 bp, Homo sapiens
gaggtgcgcggctttctgctccaggcggcccgggtgcccgctttatgcgggg

SEQ ID NO: 5 - DNA, 50 bp, Homo sapiens
tcctgctgctcccgctggttcctgcggcttctgctcagacaccaacgcca

SEQ ID NO: 6 - DNA, 45 bp, Homo sapiens
gcagggcgggtcctttgcgtccggccctctttcccctgaccataa

SEQ ID NO: 7 - DNA, 50 bp, Homo sapiens
gggccagggcttcccacgtgcgcagcaggacgcagcgctgcctgaaactc

(19) United States
(12) Patent Application Publication
(10) Pub. No.: US 2011/0300536 A1
(43) Pub. Date: Dec. 8, 2011

(54) GENE MARKER AND METHOD FOR DETECTION OF ORAL CANCER

(75) Inventors: Yu-Fen Li, Taichung (TW); Chien-Kuo Tai, Taichung (TW)

(73) Assignee: CHINA MEDICAL UNIVERSITY, Taichung (TW)

(21) Appl. No.: 12/901,219

(22) Filed: Oct. 8, 2010

(30) Foreign Application Priority Data
Jun. 3, 2010 (TW) 099117907

(51) Int. Cl. C12Q 1/68

(52) U.S. Cl. 435/6.11; 435/6.14

(57) ABSTRACT

A gene marker for the detection of oral cancer, comprising methylated CpG sites in target genes, is provided. The CpG sites in the target genes are selected from a group consisting of the following CpG sites: FLT4_E206_F, ASCL1_E24_F, KDR_E79_F, TFPI2_P9_F, TERT_E20_F, ADCYAP1_P455_R, MT1A_P49_R, and combinations thereof. A method for the detection of oral cancer, comprising the following steps is also provided: a) providing a sample to be examined from an individual; b) detecting a methylation state of at least one CpG site in a target gene on the genomic DNA from cells of the sample, wherein the CpG site in the target gene is selected from a group consisting of the following CpG sites: FLT4_E206_F, ASCL1_E24_F, KDR_E79_F, TFPI2_P9_F, TERT_E20_F, ADCYAP1_P455_R, and MT1A_P49_R; and c) determining if the individual has oral cancer based on the methylation state of the selected CpG site in the target gene.

### FIELD

The present invention relates to a gene marker for the detection of oral cancer and a method for the detection of oral cancer, and especially relates to a method for the detection of oral cancer according to the methylation state of the gene marker.

### CROSS-REFERENCES TO RELATED APPLICATIONS

This application claims priority to Taiwan Patent Application No. 099117907, filed on Jun. 3, 2010.

### BACKGROUND OF THE INVENTION

Oral cancer refers to any malignant cancerous tissue growth located in any region of the oral cavity and oropharynx and is one of the major causes of death for male Taiwanese. The risk factors of oral cancer include the usage of tobacco, alcohols, and areca nuts. Because there is a large proportion of the population chewing areca nuts in Taiwan, the incidence rate of oral cancer in Taiwan is much higher than that in other countries. Besides, the average age of death in oral cancer patients is around 50 to 60 years old which is in their golden age of life. Therefore, oral cancer, apart from reducing the lifespan of an individual, can also generate family issues and economic problems, and may have a huge impact on the Taiwanese society.

Even though there is a high recovery rate with immediate treatment, if oral cancer can be detected in the early stages, most patients do not pay special attention to the change within the oral cavity, and thus, continue the usage of areca nuts, tobacoo, and alcohols. Therefore, there are still a large number of patients who die with oral cancer each year. Accordingly, the death rate caused by oral cancer can be reduced significantly if it can be detected and treatment can be provided in the early stages.

Currently, detection methods for oral cancer include excisional biopsy, gargle and stain detection, and exfolivative cytology. Excisional biopsy is carried out by excising a suspicious lesion that is independent and obvious in position, making slices, and examining the slices with microscopy to distinguish if the testee has developed oral cancer. However, for high-risk patients whose oral mucosa has been soaked with carcinogenic areca nut juice for a long period of time, the long period of field cancerization can form diffusive precancerous lesions, and there is no single, position-independent, and clear lesion within the oral cavity, and therefore it is difficult to detect via excisional biopsy. Gargle and stain detection is carried out by using a rinse solution such as Toluidin Blue, Lugol Solution, Methylene Blue, etc. to detect oral cancer. Even though these oral dye rinse solutions are easy to use and have good sensitivity, they often get false positive results, resulting in the reduction of accuracy in detection and unnecessary waste of medical resources, and thus, the excisional biopsy detection is still needed for this method. The detection of exfolivative cytology is similar to the cervical cancer smear test. Even though exfolivative cytology is suitable for cervical cancer detection, it is not suitable for oral cancer detection. Because it often gives false negative results, the environment of the oral cavity is quite different to that of the cervix, and the detection is easy to be interfered by saliva secretion; the application and accuracy of exfolivative cytology are affected greatly. Therefore, an effective and highly accurate detection method is still required for oral cancer clinical diagnosis.

The study of the present invention is conducted according to the above demands. The inventors of the present invention found that there are seven CpG sites in seven genes within the human genomic DNA that can be used as a gene marker for the detection of oral cancer with high accuracy.

### SUMMARY OF THE INVENTION

The primary objective of this invention is to provide a gene marker for the detection of oral cancer, comprising methylated CpG sites in target genes, wherein the CpG sites in the target genes are selected from a group consisting of the following CpG sites: FLT4_E206_F, ASCL1_E24_F, KDR_E79_F, TFPI2_P9_F, TERT_E20_F, ADCYAP1_P455_R, MT1A_P49_R and combinations thereof.

Another objective of the present invention is to provide a method for the detection of oral cancer, comprising the following steps: a) providing a sample to be examined from an individual; b) detecting a methylation state of at least one CpG site in a target gene on genomic DNA from cells of the sample, wherein the CpG site in the target gene is selected from a group consisting of the following CpG sites: FLT4_E206_F, ASCL1_E24_F, KDR_E79_F, TFPI2_P9_F, TERT_E20_F, ADCYAPL1_P455_R, and MT1A_P49_R; and c) determining if the individual has oral cancer based on the methylation state of the selected CpG site in the target gene.

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

FIG. 1 is a cluster analysis dedrogram obtained by analyzing oral squamous cell carcinoma (OSCC), normal tissue adjacent to oral tumor (AN), and completely normal tissue (N) according to the degree of methylation (β value) of 1505 CpG sites;

FIGS. 2 and 3 illustrate the screening flowcharts of the gene marker of the present invention; and

FIG. 4 is a cluster analysis dedrogram obtained by analyzing oral squamous cell carcinoma (OSCC), normal tissue adjacent to oral tumor (AN), and completely normal tissue (N) according to the degree of methylation (β value) of 34 CpG sites.

### DESCRIPTION OF THE PREFERRED EMBODIMENT

Unless there is an explanation in this article otherwise, the words “a”, “an”, “the”, and other analogous words in this specification (especially in the following claims) should be considered as a singular or plural form.

The code of genomic deoxyribonucleic acid (represented as “genomic DNA” hereinafter) consists of the combination of four bases (adenine (A), thymine (T), cytosine (C), and guanine (G)) to provide a variety of genetic messages, wherein, the methylation modification of cytosine (5-methylcytosine) would affect the gene phenotype. The methylation of cytosine occurs after the synthesis of DNA, where by a DNA methyltransferase, a methyl donor, S-adenosylmethionine (SAM), transfers a methyl group to carbon 5 of cytosine. 5-methylcytosine only exists in 5′-CpG-3′ of the palindromic sequence within mammalian cells. CpG islands refer to a region of about 200 base pairs containing a large amount of CG double-nucleotides, and CpG islands are usually located near promoters of wildly expressed genes (See Gardiner-Gargen et al., CpG Islands in vertebrate genomes, Journal of Molecular Biology. July 1987; 196(2):261-282, which is entirely incorporated hereinto as reference). The methylation of cytosine in the CpG islands would promote gene silencing and stop gene expression that result in the formation of cancer.

Despite the fact that there is a correlation between cancer and the methylation modification of cytosine in the CpG islands, the degree of methylation differs in each cancer patient due to characteristics of genetic and environmental effects, and different cancers would also produce different methylation phenotypes. Furthermore, studies relating to the methylation of cytosine mostly are limited to a single or few genes, and are not adequate enough to provide the information as the basis of diagnosis. Moreover, the methylation phenotype of oral cancer is still unclear, and it is not understood which CpG site with methylation modification can detect oral cancer. Therefore, if the methylation of cytosine is intentionally used as a biomarker for the detection of oral cancer, then CpG sites that highly relate to oral cancer should be identified from the whole genomic DNA.

The inventors of the present invention found that there are seven CpG sites highly related to oral cancer within the whole genomic DNA, and they can be biomarkers for the detection of oral cancer. These seven CpG sites are from the following seven genes (referred to as the “target gene” hereinafter): FLT4, ASCL1, KDR, TFPI2, TERT, ADCYAP1, and MT1A. The names, codes, positions, and functions of these genes are represented in Table 1.

TABLE 1

| | | Gene | Chromosome | |
|---|---|---|---|---|
| Gene Code | Gene Name | Number | Position | Main Function |
| FLT4 | fms-related | 2324 | 5 | ATP binding, nucleic acid binding, |
| | tyrosine | | | protein-tyrosine kinase activity, |
| | kinase 4 | | | transferase activity, vascular |
| | | | | endothelial growth factor receptor |
| | | | | activity |
| ASCL1 | achaete-scute | 429 | 12 | Protein binding, transcription factor |
| | complex homolog 1 | | | activity |
| KDR | kinase insert | 3791 | 4 | ATP binding, nucleic acid binding, |
| | domain receptor | | | transferase activity, vascular |
| | | | | endothelial growth factor receptor |
| | | | | activity |
| TFPI2 | tissue factor | 7980 | 7 | Extracellular matrix structural |
| | pathway inhibitor 2 | | | composition, serine endopeptidase |
| | | | | inhibitor activity |
| ADCYAP1 | adenylate cyclase | 116 | 18 | Hormone activity, neuropeptide |
| | activating | | | hormone activity |
| | polypeptide 1 | | | |
| MT1A | metallothionein 1A | 4489 | 16 | Cadmium ion binding, cupper ion |
| | | | | binding, metal ion binding, zinc ion |
| | | | | binding |
| TERT | telomerase reverse | 7015 | 5 | RNA-induced DNA polymerase |
| | transcriptase | | | activity, telomeric DNA binding, |
| | | | | telomeric template RNA reverse |
| | | | | transcriptase activity, transferase |
| | | | | activity |

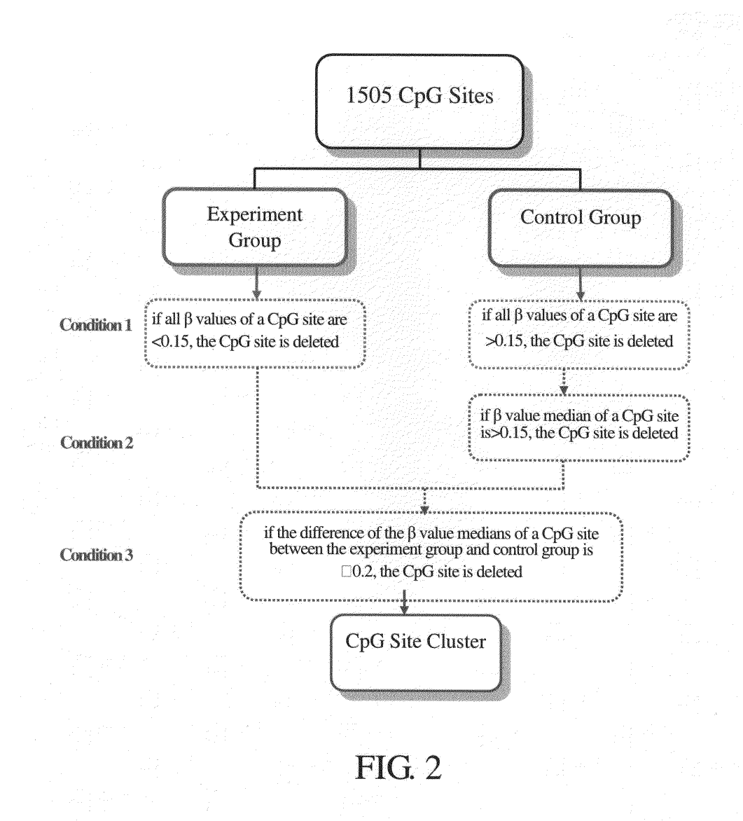

The present invention provides a gene marker for detection of oral cancer, comprising methylated CpG sites in target genes, and the CpG sites in the target genes are selected from a group consisting of the following CpG sites: FLT4_E206_F, ASCL1_E24_F, KDR_E79_F, TFPI2_P9_F, TERT_E20_F, ADCYAP1_P455_R, MT1A_P49_R, and combinations thereof. The positions of the CpG sites in the whole genomic DNA are represented in FIG. 2. Herein, if a CpG site in a target gene has been methylated, then the individual is preliminarily suspected to have oral cancer. Preferably, the gene marker of the present invention comprises the methylated CpG sites in the target genes selected from a group consisting of the following CpG sites: FLT4_E206_F, ASCL1_E24F, KDR_E79_F, TFP12_P9_F, TERT_E20_F, and combinations thereof.

TABLE 2

| | | Distance to | |
|---|---|---|---|
| | | transcription | |
| | | start site | |
| CpG Site | CpG Coordinate | (TSS) in DNA | Sequence |
| FLT4_E206_F | 180008966 | 206 | CGTGCTCCCCTCAGGCGTC[CG]CG |
| | | | CACCAGGGCCACCGTGTCCC |
| | | | (SEQ ID NO: 1) |
| ASCL1_E24_F | 101875618 | 24 | TCTGGCCAGGGAACGTGGAAGG[CG]CAC |
| | | | CGACAGGGATCCGGCCAGG |
| | | | (SEQ ID NO: 2) |
| KDR_E79_F | 55686440 | 79 | AGGCTGCCAGACGGACTTTCTGCGG[CG] |
| | | | CGCAAGTGATGCCCGGCGCAGGCAGA |
| | | | (SEQ ID NO: 3) |
| TFPI2_P9_F | 93358010 | −9 | GAGGTGCGCGGCTTTCTGCTCCAGG[CG]G |
| | | | CCCGGGTGCCCGCTTTATGCGGGG |
| | | | (SEQ ID NO: 4) |
| ADCYAP1_P455_R | 894932 | −455 | TCCTGCTGCTCCCGCTGGTTCCTG[CG]GC |
| | | | TTCTGCTCAGACACCAACGCCA |
| | | | (SEQ ID NO: 5) |
| MT1A_P49_R | 55230030 | −49 | GCAGGGCGGGTCCTTTGCGTC[CG]G |
| | | | CCCTCTTTCCCCTGACCATAA |
| | | | (SEQ ID NO: 6) |
| TERT_E20_F | 1348139 | 20 | GGGCCAGGGCTTCCCACGTG[CG]CA |
| | | | GCAGGACGCAGCGCTGCCTGAAACTC |
| | | | (SEQ ID NO: 7) |

In an embodiment of the present invention, based on the combination of one to three CpG sites, the seven CpG sites are combined and permutated, and the resulting combinations are used in the detection of oral cancer to increase the accuracy of detection. Preferably, the gene marker of the present invention comprises the methylated CpG sites in the target genes selected from a group consisting of the following CpG sites:
(1) FLT4_E206_F and ASCL1_E24_F;
(2) FLT4_E206_F, ASCL1_E24_F, and KDR_E79_F;
(3) TFPI2_P9_F, ASCL1_E24_F, and KDR_E79_F;
(4) FLT4_E206_F, TFPI2_P9_F, and ASCL1_E24_F;
(5) FLT4_E206_F and KDR_E79_F;
(6) TFPI2_P9_F and ASCL1_E24_F;
(7) FLT4_E206_F, TFPI2_P9_F, and KDR_E79_F;
(8) FLT4_E206_F and TFPI2_P9_F;
(9) FLT4_E206_F, ASCL1_E24_F, and TERT_E20_F;
(10) FLT4_E206_F;
(11) TFPI2_P9_F and KDR_E79_F;
(12) TFPI2_P9_F;
(13) ASCL1_E24_F;
(14) ADCYAP1_P455_R; and
(15) MT1A_P49_R.

In any one of the CpG site combinations from (1) to (15) above, if any one of the CpG sites of a subject has been methylated (for example, any one of “the CpG site of FLT4_E206_F” and “the CpG site of ASCL1_E24_F” is methylated in the CpG site of FLT4_E206_F and ASCL1_E24 listed in (1)), the subject can be initially determined (or suspected) to have oral cancer. Preferably, the gene marker of the present invention comprises the methylated CpG sites in the target genes selected from a group consisting of the following CpG sites:
(1) FLT4_E206_F and ASCL1_E24_F;
(2) FLT4_E206_F, ASCL1_E24_F, and KDR_E79_F;
(3) TFPI2_P9_F, ASCL1_E24_F, and KDR_E79_F;
(4) FLT4_E206_F, TFPI2_P9_F, and ASCL1_E24_F;
(5) FLT4_E206_F and KDR_E79_F;
(6) TFPI2_P9_F and ASCL1_E24_F;
(7) FLT4_E206_F, TFPI2_P9_F, and KDR_E79_F;
(8) FLT4_E206_F and TFPI2_P9_F; and
(9) FLT4_E206_F, ASCL1_E24_F, and TERT_E2O_F.

As illustrated in the following examples, the accuracy can be up to 80% or even above 90% when the gene marker of the present invention is used for the detection of oral cancer. Therefore, it can be the biomarker for effective screening of oral cancer.

Because the gene marker of the present invention may contain only one to three CpG sites (i.e., it can include a single CpG site, a combination of two CpG sites, or a combination of three CpG sites), it has excellent flexibility in application. A user can choose a suitable gene marker for screening based on the physiological characteristics and needs of the subject, and can even use various combinations of the CpG sites to conduct a full screening.

The gene marker of the present invention can be used to establish a biochip for the detection of oral cancer, such as a sensor biochip, a microprocessor biochip, a microarray biochip, etc. For example, a DNA probe can be designed based on the gene marker of the present invention, and it can be placed into a biochip, wherein a sample of the individual that has been treated by bisulfite is placed in the biochip and hybridized with the probe, and the relevant information of whether the individual has oral cancer or not can be obtained immediately, thereby, improving the efficiency of screening.

The present invention also provides a method for the detection of oral cancer, comprising the following steps: a) providing a sample to be examined from an individual; b) detecting the methylation state of at least one CpG site in a target gene on the genomic DNA from cells of the sample, wherein the CpG site in the target gene is selected from a group consisting of the following CpG sites: FLT4_E206_F, ASCL1_E24_F, KDR_E79_F, TFPI2_P9_F, TERT_E2O_F, ADCYAP1_P455_R, and MT1A_P49_R; and c) determining if the individual has oral cancer based on the methylation state of the selected CpG site in the target gene.

In step (a), a sample is obtained from the oral cavity of a testee to carry out the detection. The sample can be oral mucosal cells, oral tissue slices, saliva, blood, or combinations thereof. Preferably, the sample of the testee is collected from the oral mucosal cells, oral tissue slices, saliva, blood, or combinations thereof of the area of the suspected lesions.

In step (b), a genomic DNA of the sample is extracted to test the methylation state of at least one CpG site. The genomic DNA can be extracted with any suitable methods. For example, it can be extracted by a commercial DNA isolation kit or a DNA extraction kit

After the genomic DNA is obtained, the methylation state of the CpG sites can be analyzed in any suitable techniques for the diagnosis of oral cancer. For example, an assessment selected from a group consisting of the following can be carried out: methylation-specific PCR (MSP), quantitative methylation-specific PCR (QMSP), bisulfite sequencing (BS), microarray analysis, mass spectrometer analysis, denaturing high-performance liquid chromatography (DHPLC) analysis, pyrosequencing, and combinations thereof. In one embodiment of the present invention, the genomic DNA is first subjected to the methylation modification by bisulfite, and the methylation state of the genomic DNA is analyzed by a microarray.

Preferably, in step b) of the method of the present invention, the methylation state of at least one CpG site in the target gene selected from a group consisting of the following CpG sites is detected: FLT4_E206_F, ASCL1_E24_F, KDR_E79_F, TFPI2_P9_F, and TERT_E20_F. As described above, in the present invention, based on the combination of one to three CpG sites, the seven CpG sites can be combined and permutated to improve the accuracy of the detection. Therefore, preferably, in step b), the methylation state of the CpG sites in the target gene selected from a group consisting of the following CpG sites is detected:
(1) FLT4_E206_F and ASCL1_E24_F;
(2) FLT4_E206_F, ASCL1_E24_F, and KDR_E79_F;
(3) TFPI2_P9_F, ASCL1_E24_F, and KDR_E79_F;
(4) FLT4_E206_F, TFPI2_P9_F, and ASCL1_E24_F;
(5) FLT4_E206_F and KDR_E79_F;
(6) TFPI2_P9_F and ASCL1_E24_F;
(7) FLT4_E206_F, TFPI2_P9_F, and KDR_E79_F;
(8) FLT4_E206_F and TFPI2_P9_F;
(9) FLT4_E206_F, ASCL1_E24_F, and TERT_E20_F;
(10) FLT4_E206_F;
(11) TFPI2_P9_F and KDR_E79_F;
(12) TFPI2_P9_F;
(13) ASCL1_E24_F;
(14) ADCYAP1_P455_R; and
(15) MT1A_P49_R.

Most preferably, the methylation state of the CpG sites in the target genes selected from a group consisting of the following CpG sites is detected:
(1) FLT4_E206_F and ASCL1_E24_F;
(2) FLT4_E206_F, ASCL1_E24_F, and KDR_E79_F;
(3) TFPI2_P9_F, ASCL1_E24_F, and KDR_E79_F;
(4) FLT4_E206_F, TFPI2_P9_F, and ASCL1_E24_F;
(5) FLT4_E206_F and KDR_E79_F;
(6) TFPI2_P9_F and ASCL1_E24_F;
(7) FLT4_E206_F, TFPI2_P9_F, and KDR_E79_F;
(8) FLT4_E206_F and TFPI2_P9_F; and
(9) FLT4_E206_F, ASCL1_E24_F, and TERT_E20_F.

Finally, in step (c), according to the methylation state of the selected CpG sites, if the selected CpG sites have been methylated, then the individual is preliminarily suspected to have oral cancer.

The present invention can be optionally combined with known oral cancer detection methods to improve its application flexibility. For example, after an oral tissue slice of an individual is obtained, the detection method of the present invention as well as excisional biopsy can be carried out at the same time to further improve the accuracy of screening.

Hereinafter, the present invention will be further illustrated with reference to the following examples. However, these examples are only provided for illustrate purposes, and not to limit the scope of the present invention.

### EXAMPLE 1

### Collecting Samples

### Experiment A. Collecting Testing Samples

Samples were collected to carry out methylation analysis of gene markers, and the source of samples was from the tissue bank of China Medical University Hospital. First, oral tissues of male individuals were collected, and were classified into the experimental group (i.e., the case group) and the control group (i.e., the normal group), wherein, the experimental group was the tissue obtained by surgically resecting the tumor lesion tissue of oral cancer patients. In total, there were 40 tissue samples in the experimental group. There were a total of 15 tissue samples in the control group; 10 of which were normal tissue taken near the lesions in the oral cavities of oral cancer patients and 5 of which were normal tissue taken from the oral cavities of patients undergoing tonsillectomy.

The basic demographic characteristics of the tested male individuals are listed in Table 3. There were 40 tested male patients with oral cancer in the experimental group, and 15 tested individuals in the control group. The median age of the tested individuals in the experimental group was 53 years old (the interquartile range (IQR) was 15.5 years old), which was slightly higher than that of the control group (the median age was 48 years old, and the IQR was 25 years old). The tested individuals in the experimental group were all oral mucosa cancer patients, and according to the clinical staging TNM system of oral cancer (illustrated in Table 4), the proportion of T2 (40%) was the highest in the T stage (based on the size of the tumor), followed by T4/T4a (37.5%). The proportion of N0 (65%) was the highest in the N stage (based on the regional lymph nodes that were involved). Those in the experimental group were all considered to be in M0 of the M Stage (based on distant metastasis). Furthermore, based on the clinical stage defined by the TNM system for oral cancer, phase IV/IVa was the highest in proportion (45%), followed by phase II (30%).

TABLE 3
Basic demographic characteristics

| | Experimental | Control |
|---|---|---|
| Variables n (%) | Group | Group |
| Age (years old)□Interquatile range | 53 (15.5) | 48 (25) |
| (IQR) | | |
| Male, n (%) | 40 (100 ) | 15 (100) |
| Size of primary tumor (T Stage) | | |
| T1, n (%) | 4 (10.0) | — |
| T2, n (%) | 16 (40.0) | — |
| T3, n (%) | 5 (12.5) | — |
| T4/T4a, n (%) | 15 (37.5) | — |
| Cervical lymph node metastasis (N | | |
| Stage) | | |
| N0, n (%) | 26 (65.0) | — |
| N1, n (%) | 9 (22.5) | — |
| N2/N2b, n (%) | 5 (12.5) | — |
| Distant metastasis (M Stage) | | |
| M0, n (%) | 40 (100.0) | — |
| Clinical stage | | |
| I, n (%) | 4 (10.0) | — |
| II, n (%) | 12 (30.0) | — |
| III, n (%) | 6 (15.0) | — |
| IV/IVa, n (%) | 18 (45.0) | — |

TABLE 4
Oral Cancer Clinical Staging TNM System

| Phase | |
|---|---|
| 0 | Carcinoma in situ, tumor cells confined to the oral mucosal |
| | epithelium |
| I | The maximum diameter of tumor is less than or equal to 2 cm, |
| | and no cervical lymph node (or distant) metastasis. |
| II | The maximum diameter of tumor is greater than 2 cm but not |
| | greater than 4 cm and no cervical lymph node (or distant) |
| | metastasis. |
| III | The maximum diameter of tumor is greater than 4 cm or the |
| | tumor has been transferred to an ipsilateral cervical lymph |
| | nodes, and the maximum diameter of the lymph node is no more |
| | than 3 cm. |
| IV | 1. Tumor invades adjacent tissues (e.g., through the outer |
| | layer of bone, deep into core muscle, maxillary sinus, and skin) |
| | 2. The number of cervical lymph node metastasis is greater than |
| | one (whether it is at the ipsilateral, contralateral, or both sides |
| | of original lesion), or the diameter of lymph node is greater |
| | than 3 cm. |
| | 3. Distant metastasis has already occurred. |

The present experiment was carried out after obtaining agreement from the Research Ethic Committee, College of Public Health, China Medical University, and Human Body Experiment Committee, China Medical University Hospital.

### Experiment B. DNA Extraction

The oral tissue samples obtained from Experiment A was used as a source of genomic DNA samples and to extract DNA. A Gentra DNA isolation kit (Minneapolis, Minn., USA) was used to extract genomic DNA, and then a Zymo EZ DNA Methylation kit (Alameda, Calif., USA) was used to conduct modification treatment by sodium bisulfite. In general, cytosine (C) would change to uracil (U) after the DNA was modified with sodium bisulfate, but if methylated cytosine (i.e., 5-methylcytosine, m5C) is treated with sodium bisulfite, then the form of this base will not be changed and still stay in the form of cytosine. Therefore, a nucleotide sequencing method can be used to identify the position of methylated cytosine in a DNA sequence.

### Experiment C. Identification of Methylation

In this experiment, an Array of Illumina GoldenGate Methylation Cancer Panel II platform (San Diego, Calif., USA) was used to analyze the methylation state, and to identify the degree of methylation of 1505 CpG sites of 807 cancer related genes in the genomic DNA obtained in Experiment B. A universal primer 1 was used as the Cy3 marker to extend a non-methylated DNA template, and a universal primer 2 was used as the Cy5 marker to extend a methylated DNA template.

Then, an algorithm of methylation analysis was used to calculate the degree of methylation of every CpG site in the genomic DNA samples, and the degree of methylation was represented as the “β value.” The β value ranged from 0 to 1, and was calculated with the following formula:

β=Max(Cy5.0)/[Max(Cy3.0)+Max(Cy5.0)+100]

wherein, “Max” referred to the maximum value of the degree of methylation of a particular CpG site in the genomic DNA sample. If there are negative values for Cy5 or Cy3 then the “max” value was replaced by 0. The identification of methylation and calculation of β value described above can be seen in Bibikov et al, (2006) High-throughput DNA methylation profiling using universal bead arrays. Genome Res. 16: 383-393, which is entirely incorporated hereinto as a reference.

### Experiment D. Analysis of the Control Group Samples

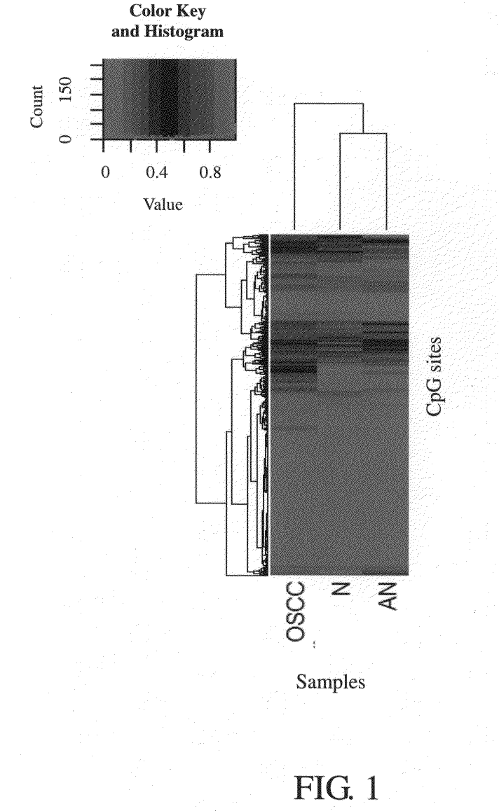

Because the samples of the control group come from two different sources of tissue (i.e., normal tissue near the lesions in the interior oral cavities of the oral cancer patients and normal tissue in the interior oral cavities of the patients receiving tonsillectomy), the β values obtained from Experiment C were used for hierarchical cluster analysis to confirm whether the two different tissue sources could be combined into a control group (normal group). Herein, the testing samples were divided into three groups, comprising oral squamous cell carcinoma (OSCC), normal tissue adjacent to the lesions of oral cancer (AN), and completely normal tissue (N). The average β values of the 1505 CpG sites of the three groups were obtained and further assessed by hierarchical cluster analysis. The analysis result is illustrated in FIG. 1, indicating that the normal tissue adjacent to the lesions of oral cancer and the completely normal tissue can be divided into the same cluster, and the merger of the two tissue sources as the control group was appropriate.

### EXAMPLE 2

### Gene Marker Screening

### Experiment E. Establishing CpG Site Cluster

First, a potential CpG site group for screening (abbreviated as “CpG site cluster” in the following descriptions) was kept, and then, the best CpG site gene markers were identified by screening. The establishment of the CpG site cluster can be classified into two stages as demonstrated in FIG. 2. The first stage was to carry out preliminary screening of 1505 CpG sites, and CpG sites that were retained belonged to the CpG site cluster where the screening selection criteria is listed below:

i) The degree of methylation (or the presence and absence of methylation) was defined by the dichotomy method. If the β value was >0.15, then the CpG site was defined as being methylated; and if the β value was <0.15, then the CpG site was defined as not being methylated. Herein, if the β values of a particular CpG site in the samples of the experimental group (i.e., the case group) were all less than 0.15 (i.e., all were non-methylated; the CpG site could not be an effective gene marker), then the CpG site would be deleted. On the contrary, if the β values of a particular CpG site in the samples of the control group (i.e., the normal group) were all greater then 0.15 (i.e., all were methylated, which belonged to normal methylation), then the CpG site would also be deleted.

ii) If the β value median of a particular CpG site in all the samples of the control group is greater than 0.15 (i.e., more than half was methylated, which belonged to normal methylation), then the CpG site would be deleted, and

iii) If the difference of β value medians of a particular CpG site between the experimental group and the control group is less than 0.2 (i.e., the difference of degree of methylation between the experimental group and the control group is too small; the CpG site could not be an effective gene marker), then the CpG site would be deleted.

The screening was carried out based on the above three conditions, and the remaining CpG sites belonged to the CpG site cluster. In condition i), 730 CpG sites could be screened out from the 1505 CpG sites; in condition ii), 604 CpG sites could be screened out from the 730 CpG sites; and in condition iii), 64 CpG sites could be screened out from the 604 CpG sites. These 64 CpG sites belonged to the CpG site cluster, covering 47 genes in total (i.e., the 64 CpG sites are from 47 different genes), and each DCC, HS3ST2, HTR1B, and NPY genes had three CpG sites reserved in the CpG site cluster.

### Experiment F. Screening and Evaluation of the CpG Site Cluster

The CpG site cluster (the 64 CpG sites) obtained in Experiment E was further screened by the following procedure to find better CpG sites, and the feasibility and accuracy of the selected CpG sites were evaluated.

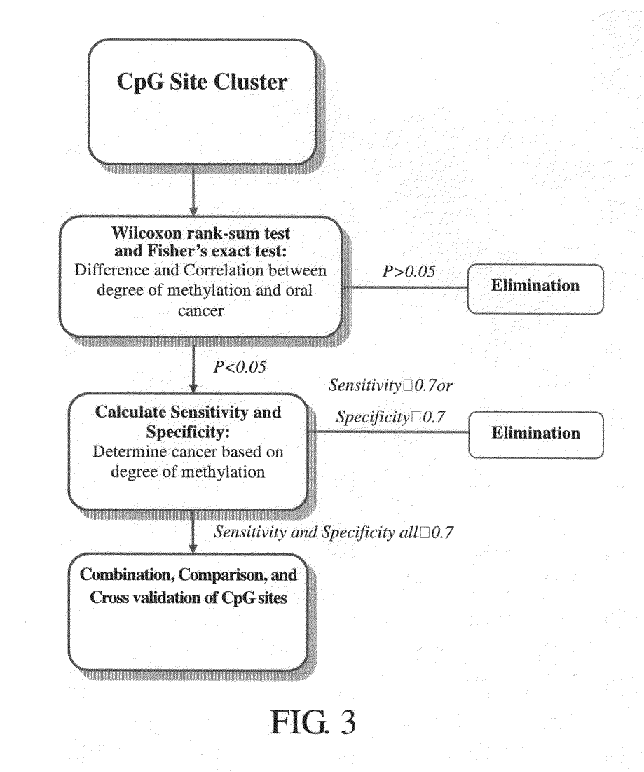

As shown in FIG. 3, the CpG sites were tested according to continuous variables and categorical variables, respectively. First, the value was considered as a continuous variable, and according to the Wilcoxon rank-sum test, CpG sites of which the degree of methylation and oral cancer had difference in statistics were screened out (Refer to: Wilcoxon, (1945), Individual comparisons by ranking methods, Biometrics Bulletin, 1, 80-83, which is entirely incorporated hereinto as reference).

Then, the degree of methylation (β value) was changed to a categorical variable of the dichotomous method (i.e., β values >0.15 were defined as being methylated, and β values <0.15 were defined as not being methylated) to conduct the Fisher's exact test to search CpG sites of which the methylation state is related to oral cancer (Fisher's exact test can be seen in Fisher, (1922), On the interpretation of X² from contingency tables, and the calculation of P. Journal of the Royal Statistical Society, 85 (1): 87-94, which is entirely incorporated hereinto as reference). If the P value of a CpG site obtained by Wilcoxon rank-sum test and Fisher's exact test was statistically significant (i.e., P<0.05), the CpG site was retained, otherwise, the CpG site would be deleted.

In the CpG site cluster (the 64 CpG sites) screened from Experiment E, the distribution of the degree of methylation (β value) and the results of Wilcoxon rank-sum test of the CpG site cluster in the experiment group and the control group are shown in Table 5. As can be seen in Table 5, the distribution of all β values of the CpG site cluster in the experiment group and the control group is significantly different (p<0.05 (significant level in statistics was set at p=0.05)), and around 86% (55/64) of the distribution of the β values of the CpG site cluster was extremely significantly different (p<0.0001). Furthermore, after the Fisher's exact test was conducted, only the CpG site of PALM2_AKAP2_P420_R was not significantly related to oral cancer and therefore, was excluded. The correlation between oral cancer and the degree of methylation of the CpG site cluster in the experiment group and control group is illustrated in Table 6.

TABLE 5
Degree of methylation (β value) of the
experimental group and the control group

| | Experimental | | Control | | |
|---|---|---|---|---|---|
| | Group | | Group | | |
| | (n = 40) | | (n = 15) | | |
| | β value | β value | β value | βvalue | |
| CpG Site | Median | (IQR) | Median | (IQR) | p value† |
| ADCYAP1_P398_F | 0.619 | (0.389) | 0.105 | (0.19) | 4.0E−06*** |
| ADCYAP1_P455_R | 0.415 | (0.319) | 0.049 | (0.087) | 4.0E−06*** |
| ASCL1_E24_F | 0.281 | (0.39) | 0.029 | (0.078) | 2.5E−05*** |
| BMP3_E147_F | 0.291 | (0.364) | 0.035 | (0.052) | 2.8E−06*** |
| BMP3_P56_R | 0.224 | (0.29) | 0.023 | (0.019) | 1.5E−05*** |
| CHGA_E52_F | 0.320 | (0.324) | 0.078 | (0.081) | 2.9E−04*** |
| CSPG2_E38_F | 0.254 | (0.313) | 0.024 | (0.037) | 2.3E−04*** |
| DBC1_P351_R | 0.446 | (0.359) | 0.119 | (0.152) | 1.2E−04*** |
| EPHA5_E158_R | 0.290 | (0.293) | 0.049 | (0.094) | 5.2E−05*** |
| EPHA7_E6_F | 0.421 | (0.432) | 0.059 | (0.08) | 7.2E−05*** |
| EPHA7_P205_R | 0.311 | (0.395) | 0.061 | (0.052) | 3.0E−05*** |
| ESR1_P151_R | 0.436 | (0.477) | 0.119 | (0.067) | 5.3E−06*** |
| EYA4_E277_F | 0.725 | (0.28) | 0.132 | (0.252) | 2.5E−05*** |
| FGF3_P171_R | 0.263 | (0.379) | 0.056 | (0.088) | 1.0E−03** |
| FLT1_P302_F | 0.252 | (0.503) | 0.022 | (0.028) | 5.2E−05*** |
| FLT1_P615_R | 0.297 | (0.459) | 0.081 | (0.119) | 3.1E−04*** |
| FLT4_E206_F | 0.342 | (0.229) | 0.061 | (0.046) | 1.9E−06*** |
| FRZB_E186_R | 0.289 | (0.49) | 0.087 | (0.07) | 1.3E−04*** |
| GABRB3_E42_F | 0.344 | (0.274) | 0.075 | (0.093) | 5.7E−05*** |
| GAS7_E148_F | 0.490 | (0.594) | 0.057 | (0.059) | 2.7E−05*** |
| GDF10_P95_R | 0.413 | (0.571) | 0.086 | (0.208) | 3.8E−03** |
| GPX3_E178_F | 0.331 | (0.37) | 0.042 | (0.062) | 3.0E−05*** |
| HHIP_E94_F | 0.293 | (0.635) | 0.041 | (0.074) | 2.7E−03** |
| HHIP_P307_R | 0.458 | (0.434) | 0.144 | (0.09) | 8.8E−04*** |
| HOXA5_P1324_F | 0.254 | (0.149) | 0.050 | (0.049) | 3.0E−06*** |
| HOXB13_P17_R | 0.422 | (0.411) | 0.069 | (0.063) | 3.8E−05*** |
| HS3ST2_P171_F | 0.605 | (0.368) | 0.090 | (0.164) | 5.3E−06*** |
| HS3ST2_P546_F | 0.319 | (0.265) | 0.076 | (0.098) | 1.1E−05*** |
| HTR1B_P107_F | 0.439 | (0.354) | 0.106 | (0.123) | 1.2E−05*** |
| ICA1_P72_R | 0.298 | (0.443) | 0.077 | (0.032) | 7.7E−04*** |
| IHH_E186_F | 0.452 | (0.47) | 0.076 | (0.1) | 4.1E−04*** |
| ISL1_E87_R | 0.328 | (0.429) | 0.058 | (0.123) | 5.1E−04*** |
| ISL1_P379_F | 0.435 | (0.499) | 0.090 | (0.132) | 4.4E−04*** |
| KDR_E79_F | 0.250 | (0.311) | 0.040 | (0.058) | 7.2E−05*** |
| KDR_P445_R | 0.289 | (0.39) | 0.040 | (0.045) | 8.2E−06*** |
| MME_E29_F | 0.631 | (0.685) | 0.135 | (0.095) | 2.1E−03** |
| MME_P388_F | 0.286 | (0.373) | 0.052 | (0.146) | 1.9E−03** |
| MT1A_P49_R | 0.419 | (0.361) | 0.053 | (0.064) | 3.7E−06*** |
| MYH11_P22_F | 0.381 | (0.454) | 0.031 | (0.083) | 4.4E−06*** |
| MYOD1_E156_F | 0.461 | (0.21) | 0.087 | (0.067) | 5.0E−07*** |
| NPY_E31_R | 0.379 | (0.446) | 0.078 | (0.078) | 1.4E−05*** |
| NPY_P295_F | 0.624 | (0.292) | 0.121 | (0.184) | 6.3E−06*** |
| NPY_P91_F | 0.233 | (0.254) | 0.020 | (0.048) | 4.4E−06*** |
| NTRK3_E131_F | 0.276 | (0.39) | 0.026 | (0.023) | 2.0E−04*** |
| NTRK3_P752_F | 0.356 | (0.386) | 0.042 | (0.063) | 7.5E−06*** |
| OPCML_E219_R | 0.346 | (0.386) | 0.083 | (0.078) | 5.8E−04*** |
| PALM2_AKAP2_P420_R | 0.405 | (0.52) | 0.130 | (0.175) | 2.3E−02* |
| PEG10_P978_R | 0.363 | (0.337) | 0.146 | (0.165) | 1.5E−03** |
| PENK_P447_R | 0.465 | (0.286) | 0.089 | (0.147) | 9.8E−07*** |
| RASGRF1_E16_F | 0.381 | (0.378) | 0.029 | (0.077) | 6.3E−06*** |
| SLC5A8_E60_R | 0.461 | (0.386) | 0.139 | (0.157) | 7.2E−05*** |
| SLC5A8_P38_R | 0.390 | (0.488) | 0.128 | (0.131) | 1.9E−03** |
| SLIT2_E111_R | 0.365 | (0.467) | 0.058 | (0.035) | 3.0E−05*** |
| SLIT2_P208_F | 0.386 | (0.498) | 0.087 | (0.078) | 4.4E−04*** |
| ST6GAL1_P528_F | 0.277 | (0.486) | 0.073 | (0.105) | 8.2E−03** |
| TERT_E20_F | 0.300 | (0.265) | 0.028 | (0.074) | 7.5E−06*** |
| TERT_P360_R | 0.531 | (0.328) | 0.072 | (0.118) | 5.3E−06*** |
| TFPI2_P152_R | 0.385 | (0.408) | 0.064 | (0.085) | 5.2E−05*** |
| TFPI2_P9_F | 0.305 | (0.382) | 0.033 | (0.037) | 9.8E−06*** |
| TMEFF2_E94_R | 0.319 | (0.34) | 0.024 | (0.056) | 9.2E−05*** |
| TMEFF2_P152_R | 0.500 | (0.399) | 0.091 | (0.089) | 3.8E−05*** |
| TPEF_seq_44_S88_R | 0.472 | (0.285) | 0.108 | (0.112) | 2.5E−06*** |
| WNT2_E109_R | 0.254 | (0.37) | 0.019 | (0.027) | 4.4E−04*** |
| WT1_E32_F | 0.643 | (0.391) | 0.093 | (0.151) | 6.3E−06*** |

†Wilcoxon rank-sum test,
*p < 0.05,
**p < 0.01,
***p < 0.001

TABLE 6
Correlation between Methylation of CpG Sites and Oral Cancer

| | Case Group | Control Group | | |
|---|---|---|---|---|
| | (n = 40) | (n = 15) | Odds | (95% |
| | Methylated | Methylated | Ratios | Confidence |
| CpG Site | n (%) | n (%) | (ORs) | Intervals (CI)) |
| ADCYAP1_P398_F | 37 (92.5) | 6 (40) | 18.50 | (3.87-88.54) |
| ADCYAP1_P455_R | 35 (87.5) | 2 (13.33) | 45.50 | (7.84-264.22) |
| ASCL1_E24_F | 30 (75) | 0 (0) | —‡ | —‡ |
| BMP3_E147_F | 30 (75) | 2 (13.33) | 19.50 | (3.74-101.72) |
| BMP3_P56_R | 26 (65) | 1 (6.67) | 26.00 | (3.09-218.84) |
| CHGA_E52_F | 31 (77.5) | 3 (20) | 13.78 | (3.18-59.73) |
| CSPG2_E38_F | 26 (65) | 1 (6.67) | 26.00 | (3.09-218.84) |
| DBC1_P351_R | 33 (82.5) | 5 (33.33) | 9.43 | (2.45-36.3) |
| EPHA5_E158_R | 33 (82.5) | 2 (13.33) | 30.64 | (5.61-167.31) |
| EPHA7_E6_F | 32 (80) | 2 (13.33) | 26.00 | (4.85-139.26) |
| EPHA7_P205_R | 31 (77.5) | 2 (13.33) | 22.39 | (4.24-118.15) |
| ESR1_P151_R | 36 (90) | 3 (20) | 36.00 | (7.03-184.35) |
| EYA4_E277_F | 35 (87.5) | 6 (40) | 10.50 | (2.6-42.35) |
| FGF3_P171_R | 28 (70) | 1 (6.67) | 32.67 | (3.85-277.23) |
| FLT1_P302_F | 27 (67.5) | 0 (0) | — | — |
| FLT1_P615_R | 28 (70) | 5 (33.33) | 4.67 | (1.31-16.6) |
| FLT4_E206_F | 33 (82.5) | 0 (0) | — | — |
| FRZB_E186_R | 28 (70) | 2 (13.33) | 15.17 | (2.96-77.8) |
| GABRB3_E42_F | 33 (82.5) | 3 (20) | 18.86 | (4.19-84.96) |
| GAS7_E148_F | 30 (75) | 1 (6.67) | 42.00 | (4.89-361.03) |
| GDF10_P95_R | 27 (67.5) | 5 (33.33) | 4.15 | (1.18-14.66) |
| GPX3_E178_F | 30 (75) | 2 (13.33) | 19.50 | (3.74-101.72) |
| HHIP_E94_F | 26 (65) | 2 (13.33) | 12.07 | (2.38-61.26) |
| HHIP_P307_R | 32 (80) | 7 (46.67) | 4.57 | (1.28-16.38) |
| HOXA5_P1324_F | 32 (80) | 2 (13.33) | 26.00 | (4.85-139.26) |
| HOXB13_P17_R | 32 (80) | 3 (20) | 16.00 | (3.63-70.54) |
| HS3ST2_P171_F | 36 (90) | 4 (26.67) | 24.75 | (5.3-115.64) |
| HS3ST2_P546_F | 34 (85) | 4 (26.67) | 15.58 | (3.71-65.53) |
| HTR1B_P107_F | 34 (85) | 5 (33.33) | 11.33 | (2.85-45.07) |
| ICA1_P72_R | 27 (67.5) | 1 (6.67) | 29.08 | (3.44-245.64) |
| IHH_E186_F | 30 (75) | 3 (20) | 12.00 | (2.8-51.34) |
| ISL1_E87_R | 30 (75) | 4 (26.67) | 8.25 | (2.14-31.82) |
| ISL1_P379_F | 30 (75) | 4 (26.67) | 8.25 | (2.14-31.82) |
| KDR_E79_F | 28 (70) | 0 (0) | — | — |
| KDR_P445_R | 26 (65) | 1 (6.67) | 26.00 | (3.09-218.84) |
| MME_E29_F | 31 (77.5) | 5 (33.33) | 6.89 | (1.87-25.41) |
| MME_P388_F | 29 (72.5) | 4 (26.67) | 7.25 | (1.9-27.64) |
| MT1A_P49_R | 34 (85) | 2 (13.33) | 36.83 | (6.57-206.36) |
| MYH11_P22_F | 31 (77.5) | 2 (13.33) | 22.39 | (4.24-118.15) |
| MYOD1_E156_F | 36 (90) | 3 (20) | 36.00 | (7.03-184.35) |
| NPY_E31_R | 33 (82.5) | 2 (13.33) | 30.64 | (5.61-167.31) |
| NPY_P295_F | 38 (95) | 7 (46.67) | 21.71 | (3.79-124.54) |
| NPY_P91_F | 27 (67.5) | 1 (6.67) | 29.08 | (3.44-245.64) |
| NTRK3_E131_F | 24 (60) | 0 (0) | —‡ | —‡ |
| NTRK3_P752_F | 32 (80) | 2 (13.33) | 26.00 | (4.85-139.26) |
| OPCML_E219_R | 27 (67.5) | 2 (13.33) | 13.50 | (2.65-68.84) |
| PEG10_P978_R | 33 (82.5) | 7 (46.67) | 5.39 | (1.47-19.8) |
| PENK_P447_R | 35 (87.5) | 4 (26.67) | 19.25 | (4.39-84.49) |
| RASGRF1_E16_F | 33 (82.5) | 2 (13.33) | 30.64 | (5.61-167.31) |
| SLC5A8_E60_R | 34 (85) | 6 (40) | 8.50 | (2.21-32.76) |
| SLC5A8_P38_R | 29 (72.5) | 5 (33.33) | 5.27 | (1.47-18.93) |
| SLIT2_E111_R | 26 (65) | 1 (6.67) | 26.00 | (3.09-218.84) |
| SLIT2_P208_F | 27 (67.5) | 2 (13.33) | 13.50 | (2.65-68.84) |
| ST6GAL1_P528_F | 27 (67.5) | 3 (20) | 8.31 | (1.99-34.64) |
| TERT_E20_F | 30 (75) | 1 (6.67) | 42.00 | (4.89-361.03) |
| TERT_P360_R | 34 (85) | 4 (26.67) | 15.58 | (3.71-65.53) |
| TFPI2_P152_R | 33 (82.5) | 3 (20) | 18.86 | (4.19-84.96) |
| TFPI2_P9_F | 31 (77.5) | 0 (0) | — | — |
| TMEFF2_E94_R | 27 (67.5) | 1 (6.67) | 29.08 | (3.44-245.64) |
| TMEFF2_P152_R | 35 (87.5) | 5 (33.33) | 14.00 | (3.37-58.21) |
| TPEF_seq_44_S88_R | 35 (87.5) | 5 (33.33) | 14.00 | (3.37-58.21) |
| WNT2_E109_R | 25 (62.5) | 1 (6.67) | 23.33 | (2.78-195.83) |
| WT1_E32_F | 37 (92.5) | 7 (46.67) | 14.10 | (2.98-66.64) |

‡Odds ratios (ORs) and 95% confidence intervals (CI) cannot be calculated due to partial blank data (number of people is 0)

Finally, the sensitivity and specificity of the CpG sites were calculated. The degree of methylation was defined by the dichotomous method (i.e., β values >0.15 were defined as being methylated, and β values <0.15 was defined as not being methylated) to predict if the tested individual has oral cancer and calculate the sensitivity and specificity of each CpG site thereby, wherein a CpG site of which the sensitivity and specificity were both above 0.7 was retained, otherwise, the CpG site would be deleted.

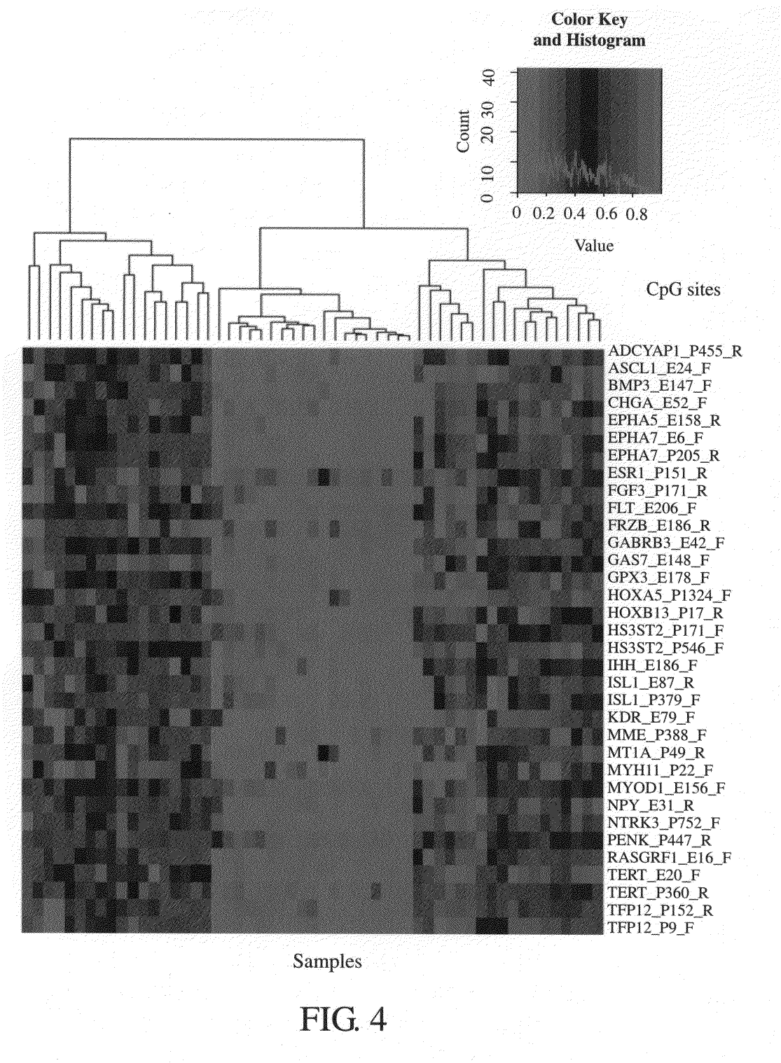

After the above screening steps, a total of 34 CpG sites were obtained and their sensitivity and specificity were all >0.7. These 34 CpG sites were from 29 genes, and the functions of these genes can be classified into 11 categories: signal transduction, tumor inhibition, differentiation, metabolism, blood coagulation, cell cycle, DNA repairing, imprinting, cell adhesion, cell development, and hematopoiesis. The cluster distribution analysis of these 34 CpG sites is shown in FIG. 4, showing that the tested samples could be divided into three clusters, where the left and right sides of the clusters are mainly oral squamous cell carcinoma (OSCC), and the middle clusters are mainly normal tissue adjacent to the lesions of oral cancer (AN) and completely normal tissue (N).

### Experiment G. Combination, Comparison, and Cross Validation of CpG Sites

Statistical Analysis System (SAS) software (9.2 version) was used to calculate the area under curve (AUC) of Receiver Operator Characteristic (ROC) curve to evaluate the accuracy, wherein as the AUC value increases, the accuracy also increases. The magnitude of the AUC value was used to sequence the 34 CpG sites of which the sensitivity and specificity are both above 0.7 in Experiment F. Herein, the top ten CpG sites were combined randomly, including combinations of one, two or three CpG sites. After combination and permutation, the sensitivity and specificity of each combination for predicting oral cancer (if any one of the CpG sites in each combination was methylated, then the subject was considered (or suspected) to have oral cancer) were calculated again, and cross validation was conducted to test the accuracy (including the average probability of correct classification and standard error) to select the best combinations of CpG sites.

The SAS software was used for the statistics analysis in Experiment D to Experiment G., and the R package software (version 2.8.1) was used as the drawing tool for heat maps (FIGS. 1 and 4). The SAS macro CVLR software written by Clint Moore was used for 5-fold cross validation (repeated 5000 times) to calculate the average probability of correct classification and standard error of each CpG site combination.

The accuracy of each CpG site combination for screening oral cancer is illustrated in Table 7, and the rank in the table was sequenced according to the AUC value first, followed by a combination containing minimum CpG sites, and the average probability of correct classification calculated by cross validation. Because there are a large number of combinations, only the top 15 are listed in Table 7 (including the top 5 in combinations of single CpG site, two CpG sites, and three CpG sites). The top 15 combinations contain seven CpG sites in total: FLT4_E206_F, ASCL1_E24_F, KDR_E79_F, TFPI2_P9_F, ADCYAP1_P455_R, MT1A_P49_R, and TERT_E20_F.

TABLE 7
The Accuracy of Each Combination of CpG Sites for Oral Cancer Screening

| | | | | | Average | |
|---|---|---|---|---|---|---|
| | | | | | Probability | |
| | | | | AUC | of Correct | Standard |
| Rank | Combination of CpG Sites | Specificity | Sensitivity | value | Classification | Error |
| 1 | FLT4_E206_F + | 1 | 0.900 | 0.950 | 0.926 | 0.001 |
| | ASCL1_E24_F | | | | | |
| 2 | FLT4_E206_F + | 1 | 0.900 | 0.950 | 0.928 | 0.001 |
| | ASCL1_E24_F + KDR_E79_F | | | | | |
| 3 | TFPI2_P9_F + ASCL1_E24_F + | 1 | 0.900 | 0.950 | 0.927 | 0.001 |
| | KDR_E79_F | | | | | |
| 4 | FLT4_E206_F + TFPI2_P9_F + | 1 | 0.900 | 0.950 | 0.927 | 0.001 |
| | ASCL1_E24_F | | | | | |
| 5 | FLT4_E206_F + KDR_E79_F | 1 | 0.875 | 0.938 | 0.910 | 0.001 |
| 6 | TFPI2_P9_F + ASCL1_E24_F | 1 | 0.875 | 0.938 | 0.908 | 0.001 |
| 7 | FLT4_E206_F + TFPI2_P9_F + | 1 | 0.875 | 0.938 | 0.911 | 0.001 |
| | KDR_E79_F | | | | | |
| 8 | FLT4_E206_F + TFPI2_P9_F | 1 | 0.850 | 0.925 | 0.891 | 0.001 |
| 9 | FLT4_E206_F + | 0.933 | 0.900 | 0.917 | 0.912 | 0.001 |
| | ASCL1_E24_F + TERT_E20_F | | | | | |
| 10 | FLT4_E206_F | 1 | 0.825 | 0.913 | 0.872 | 0.001 |
| 11 | TFPI2_P9_F + KDR_E79_F | 1 | 0.825 | 0.913 | 0.876 | 0.001 |
| 12 | TFPI2_P9_F | 1 | 0.775 | 0.888 | 0.828 | 0.002 |
| 13 | ASCL1_E24_F | 1 | 0.750 | 0.875 | 0.800 | 0.002 |
| 14 | ADCYAP1_P455_R | 0.867 | 0.875 | 0.871 | 0.872 | 0.001 |
| 15 | MT1A_P49_R | 0.867 | 0.850 | 0.858 | 0.853 | 0.001 |

Table 4 shows that the screening accuracy of 85%, even above 90% can be provided when the gene marker of the present invention was used. Therefore, the gene marker can be used as an effective biomarker for the detection of oral cancer.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. A method for detection of oral cancer, comprising the following steps:
a) providing a sample to be examined from an individual;
b) detecting a methylation state of at least one CpG site in a target gene on genomic DNA from cells of the sample, wherein the CpG site in the target gene is selected from a group consisting of FLT4_E206_F, ASCL1_E24_F, KDR_E79_F, TFPI2_P9_P9_F, TERT_E20_F, ADCYAP1_P455_R, and MT1A_P49_R; and
c) determining if the individual has oral cancer based on the methylation state of the selected CpG site in the target gene.

2. The method as claimed in claim 1, wherein the sample in step a) is selected from a group consisting of oral mucosal cells, oral tissue slices, saliva, blood, and combinations thereof.

3. The method as claimed in claim 1, wherein step b) comprises carrying out an assessment selected from a group consisting of methylation-specific PCR (MSP), quantitative methylation-specific PCR (QMSP), bisulfite sequencing (BS), microarray, mass spectrometer, denaturing high-performance liquid chromatography (DHPLC), pyrosequencing, and combinations thereof.

4. The method as claimed in claim 1, wherein in step b), a methylation state of a CpG site selected from a group consisting of the following is detected: FLT4_E206_F, ASCL1_E24_F, KDR_E79_F, TFPI2_P9_F, TERT_E20_F, and combinations thereof.

5. The method as claimed in claim 1, wherein in step b), a methylation state of a CpG site selected from a group consisting of the following is detected:
(1) FLT4_E206_F and ASCL1_E24_F;
(2) FLT4_E206_F, ASCL1_E24_F, and KDR_E79_F;
(3) TFPI2_P9_F, ASCL1_E24_F, and KDR_E79_F;
(4) FLT4_E206_F, TFPI2_P9_F, and ASCL1_E24_F;
(5) FLT4_E206_F and KDR_E79_F;
(6) TFPI2_P9_F and ASCL1_E24_F;
(7) FLT4_E206_F, TFPI2_P9_F, and KDR_E79_F;
(8) FLT4_E206_F and TFPI2_P9_F;
(9) FLT4_E206_F, ASCL1_E24_F, and TERT_E20_F;
(10) FLT4_E206_F;
(11) TFPI2_P9_F and KDR_E79_F;
(12) TFPI2_P9_F;
(13) ASCL1_E24_F;
(14) ADCYAP1_P455_R; and
(15) MT1A_P49_R.

6. The method as claimed in claim 5, wherein in step b), a methylation state of a CpG site selected from a group consisting of the following is detected:
(1) FLT4_E206_F and ASCL1_E24_F;
(2) FLT4_E206_F, ASCL1_E24_F, and KDR_E79_F;
(3) TFPI2_P9_F, ASCL1_E24_F, and KDR_E79_F;
(4) FLT4_E206_F, TFPI2_P9_F, and ASCL1_E24_F;
(5) FLT4_E206_F and KDR_E79_F;
(6) TFPI2_P9_F and ASCL1_E24_F;
(7) FLT4_E206_F, TFPI2_P9_F, and KDR_E79_F;
(8) FLT4_E206_F and TFPI2_P9_F; and
(9) FLT4_E206_F, ASCL1_E24_F, and TERT_E20_F.
